(19) United States
(12) Patent Application Publication
(10) Pub. No.: US 2008/0287799 A1
(43) Pub. Date: Nov. 20, 2008

(54) Method and apparatus for measuring volumetric flow

(75) Inventors: Anne Lindsay Hall, New Berlin, WI (US); Jonathan Matthews Rubin, Ann Arbor, MI (US); J. Brian Fowlkes, Ann Arbor, MI (US); Oliver Daniel Kripfgans, Ann Arbor, MI (US); Paul L. Carson, Ann Arbor, MI (US)

Correspondence Address: DEAN D. SMALL;THE SMALL PATENT LAW GROUP LLP, 225 S. MERAMEC, STE. 725T, ST. LOUIS, MO, 63105, US

(73) Assignees: GENERAL ELECTRIC COMPANY; MICHIGAN, REGENTS OF THE UNIVERSITY OF, THE

(21) Appl. No.: 11/803,966

(22) Filed: May 16, 2007

(51) Int. Cl. A61B 8/06

(52) U.S. Cl. 600/454

(57) ABSTRACT

An ultrasound system comprises an ultrasound probe, a user interface and a processor. The ultrasound probe comprises a transducer face emitting ultrasound beams into a patient. The probe acquires a volume of ultrasound data comprising a blood vessel. The user interface defines a surface on an image that is based on the volume. The surface bisects the blood vessel and further comprises a plurality of points where at least some of the points are located at unequal distances with respect to the transducer face. The processor is configured to steer a subset of the ultrasound beams to intersect the surface at a 90 degree angle and calculate volumetric flow information through the blood vessel based on the ultrasound data corresponding to the surface.

### BACKGROUND OF THE INVENTION

This invention relates generally to ultrasound imaging, and more particularly, to measuring volumetric flow through a vessel.

Ultrasound Doppler imaging is commonly used to detect the presence of blood flow in the body, but not to quantitatively measure the blood flow. Flow velocities at a given point in the vessel can be estimated using the measured Doppler shift and correcting for the relative angle between the ultrasound firing and the vessel orientation. Even so, the calculation of true volume flow cannot be performed without making assumptions regarding the vessel geometry and the flow profile within the vessel. The most common method for estimating volume flow is performed by multiplying the mean spatial velocity imaged within the vessel by the vessel cross-sectional area. In this method, the vessel cross-sectional area is estimated by assuming a circular vessel cross-section and non-spatial variation of the flow within that cross-sectional area.

Transducer elements within an ultrasound probe transmit ultrasound signals into the body. The transducer elements form a transducer face. Methods under current development define a plane that is equidistant from the transducer face of the ultrasound probe and the blood flow is measured through the plane. The plane matches the outer geometry of the transducer face, for example, curved or straight, and the orientation of the plane is limited to be parallel with respect to the transducer face. Thus, the computed volume flow estimates are orthogonal to the transducer face. The orientation of the plane, however, may not coincide with a desired orientation for measuring the flow through the anatomy of interest and thus the user may need to scan from different angles to locate an optimum orientation for the anatomy that matches the orientation of the transducer face.

Therefore, a need exists for calculating volumetric blood flow through a vessel without limiting the plane through the anatomy by the probe orientation and outer geometry.

### BRIEF DESCRIPTION OF THE INVENTION

In one embodiment, an ultrasound system comprises an ultrasound probe, a user interface and a processor. The ultrasound probe comprises a transducer face emitting ultrasound beams into a patient. The probe acquires a volume of ultrasound data comprising a blood vessel. The user interface defines a surface on an image that is based on the volume. The surface bisects the blood vessel and further comprises a plurality of points where at least some of the points are located at unequal distances with respect to the transducer face. The processor is configured to steer a subset of the ultrasound beams to intersect the surface at a 90 degree angle and calculate volumetric flow information through the blood vessel based on the ultrasound data corresponding to the surface.

In another embodiment, a method for calculating volumetric flow information through a vessel comprises acquiring a volume of ultrasound data with an ultrasound probe. The volume comprises a vessel and the ultrasound probe comprises a transducer face for emitting and receiving ultrasound beams. First and second surfaces are defined within the volume of ultrasound data. The first and second surfaces intersect the vessel and are formed equidistant from each other. An average volumetric flow through the vessel is calculated based on the ultrasound data corresponding to the first and second surfaces.

In another embodiment, a method for calculating a volume of flow through a vessel comprises acquiring a volume of ultrasound data with an ultrasound probe. The volume comprises a vessel and the ultrasound probe comprises a transducer face for emitting and receiving ultrasound beams. A first surface is defined on an image based on the volume. The first surface bisects the vessel and the first surface further comprises a plurality of points where at least a portion of the points are at different distances from the transducer face. A first subset of the ultrasound beams is steered to intersect the first surface at a 90 degree angle, and a first volume of flow is calculated based on the ultrasound data corresponding to the first surface.

### BRIEF DESCRIPTION OF THE DRAWINGS

FIG. 1 illustrates a block diagram of an ultrasound system formed in accordance with an embodiment of the present invention.

FIG. 2 is a block diagram of a handheld or hand carried ultrasound imaging device formed in accordance with an embodiment of the present invention.

FIG. 3 illustrates an example of calculating volume of flow through a vessel based on at least two surfaces that are parallel to each other in accordance with an embodiment of the present invention.

FIG. 4 illustrates an example of using a surface that is not parallel with respect to the transducer face to determine the amount of blood moving through a particular vessel in accordance with an embodiment of the present invention.

FIG. 5 illustrates a method for calculating volumetric flow information through a vessel in accordance with an embodiment of the present invention.

FIG. 6 illustrates an example of using multiple surfaces to detect multiple volumetric flow values at different points within the same volume of data in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

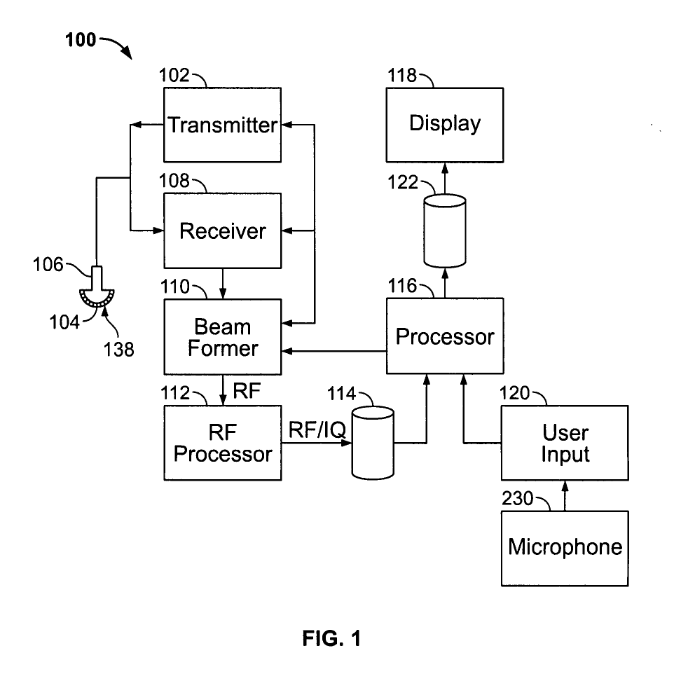

FIG. 1 illustrates a block diagram of an ultrasound system 100. The ultrasound system 100 includes a transmitter 102 that drives transducer elements 104 within a probe 106 to emit pulsed ultrasonic signals into a body. A variety of geometries may be used including 2D probes capable of scanning volumes over time. The transducer elements 104 form a transducer face 138. The ultrasonic signals may be formed in beams that are emitted from the transducer elements 104 along the transducer face 138. For example, during beamforming a subset of transducer elements 104 are activated to form an ultrasound beam. The subset of transducer elements 104 used for a first ultrasound beam may be different than the subsets used for other ultrasound beams, although some overlap may exist. In one embodiment, the ultrasound beams are emitted having a 90 degree transmission angle with respect to the transducer face 138. In another embodiment, the ultrasound beams may be steered or directed based on the scanning mode and thus have transmission angles other than 90 degrees with respect to the transducer face 138.

The ultrasonic signals are back-scattered from structures in the body, like blood cells or muscular tissue, to produce echoes that return to the transducer elements 104. The echoes are received by a receiver 108. The received echoes are passed through a beamformer 110 that performs beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to an RF/IQ buffer 114 for temporary storage.

A volume or multi-dimensional dataset of ultrasound information may be obtained by various techniques, including, for example, real-time imaging, volume scanning, scanning with transducers having positioning sensors, freehand scanning using a voxel correlation technique, scanning with matrix array transducers, and the like. The position of each echo signal sample (voxel) is defined in terms of geometrical accuracy (i.e., the distance from one voxel to the next), ultrasonic response, and optionally, derived values from the ultrasonic response. Typical ultrasonic responses include gray scale values, color flow values, and angio or power Doppler information, although others are also possible.

A user input 120 may be used to control operation of the ultrasound system 100, including, to control the input of patient data, scan parameters and/or to change a scanning mode, identification of one or more surfaces within an image that is used to determine a volume of flow through anatomy, and the like. Various embodiments may be configured for controlling the ultrasound system 100, such as by including a set of user controls that may be provided, for example, as part of a touch screen or panel, and as manual inputs, such as user operable switches, buttons, and the like. User control may also include using voice commands provided via a microphone 230.

The ultrasound system 100 includes a processor 116 to process the acquired ultrasound information (i.e., RF signal data or IQ data pairs) and prepare frames of ultrasound information for display on display 118. The processor 116 is adapted to perform one or more processing operations according to a plurality of selectable ultrasound modalities on the acquired ultrasound information. Acquired ultrasound information may be processed in real-time during a scanning session as the echo signals are received.

The ultrasound system 100 may continuously acquire ultrasound information at a frame rate that exceeds fifty frames per second, which is the approximate perception rate of the human eye. The acquired ultrasound information is displayed on the display 118 at a slower frame-rate. A memory 122 optionally is included for storing processed frames of acquired ultrasound information that are not scheduled to be displayed immediately. In an exemplary embodiment, the memory 122 is of sufficient capacity to store at least several seconds worth of frames of ultrasound information. The frames of ultrasound information are stored in a manner to facilitate retrieval thereof according to an order or time of acquisition. The memory 122 may comprise any known data storage medium.

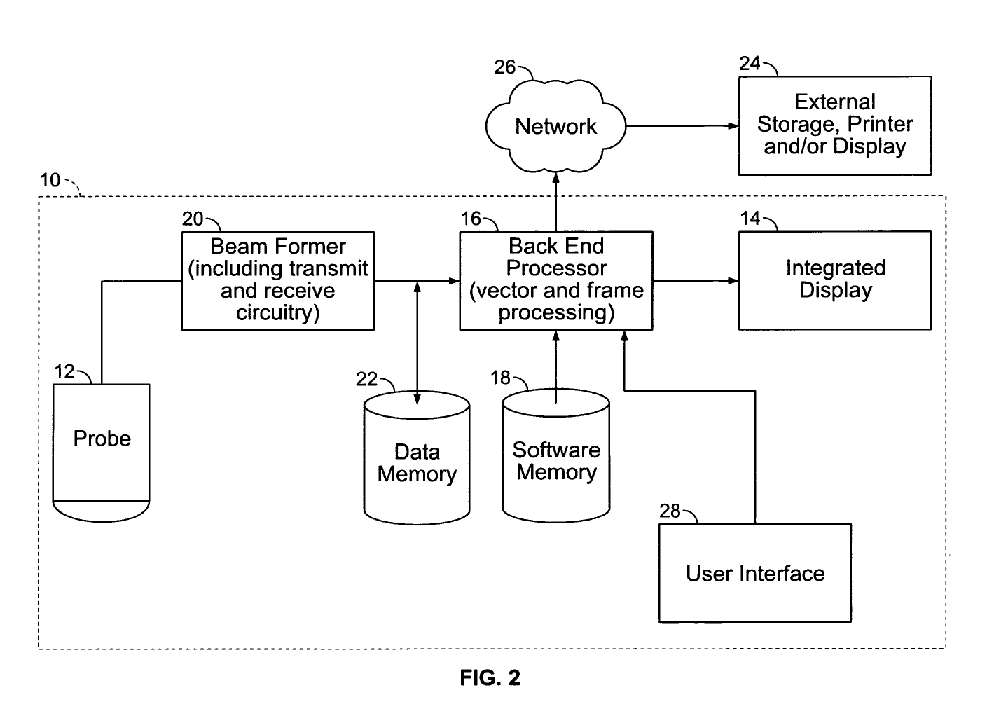

FIG. 2 is a block diagram of a handheld or hand carried ultrasound imaging device 10 having a probe 12 configured to acquire ultrasonic data. Therefore, the hand carried ultrasound imaging device 10 is easily portable by the user or operator. An integrated display 14 (e.g., an internal display) is also provided and is configured to display a medical image. A data memory 22 stores acquired image data that may be processed by a beamformer 20 in some embodiments of the present invention.

To display a medical image using the probe 12, a back end processor 16 is provided with a software or firmware memory 18 containing instructions to perform frame processing, scan conversion, and resolution selection using acquired ultrasonic image data from the probe 12, possibly further processed by the beamformer 20 in some configurations. Dedicated hardware may be used instead of software for performing scan conversion, or a combination of dedicated hardware and software, or software in combination with a general purpose processor or a digital signal processor.

Software or firmware memory 18 may comprise a read only memory (ROM), random access memory (RAM), a miniature hard drive, a flash memory card, or any kind of device (or devices) configured to read instructions from a machine-readable medium or media. The instructions contained in software or firmware memory 18 further include instructions to produce a medical image of suitable resolution for display on integrated display 14, and to send image data stored in a data memory 22 to an external device 24. The ultrasonic data itself may be sent from back end processor 16 to external device 24 via a wired or wireless network (or direct connection, for example, via a serial or parallel cable or USB port) 26 under control of processor 16 and user interface 28. In some embodiments, external device 24 may be a computer or a workstation having a display. Alternatively, external device 24 may be a separate external display or a printer capable of receiving image data from the hand carried ultrasound imaging device 10 and of displaying or printing images (that may have greater resolution than the integrated display 14).

A user interface 28 (that may also include integrated display 14) is provided to receive commands from an operator. The commands may instruct back end processor 16 to display the acquired image data on integrated display 14, adjust scan parameters, define a surface within the image for calculating volumetric flow through a vessel and send the acquired image data to the external device 24 in the same or a higher resolution than that displayable on integrated display 14.

The handheld or hand carried ultrasound imaging device 10 may be, for example, a miniaturized ultrasound system. As used herein, “miniaturized” means that the ultrasound system is a handheld or hand-carried device or is configured to be carried in a person's hand, pocket, briefcase-sized case, or backpack. For example, the ultrasound system 10 may be a hand-carried device having a size of a typical laptop computer, for instance, having dimensions of approximately 2.5 inches in depth, approximately 14 inches in width, and approximately 12 inches in height. The ultrasound system 10 may weigh about ten pounds.

As another example, the ultrasound system 10 may be a pocket-sized ultrasound system. By way of example, the pocket-sized ultrasound system may be approximately 2 inches wide, approximately 4 inches in length, and approximately 0.5 inches in depth and weigh less than 3 ounces. The pocket-sized ultrasound system may include a display, a user interface (i.e., keyboard) and an input/output (I/O) port for connection to the probe (all not shown). It should be noted that the various embodiments may be implemented in connection with a miniaturized ultrasound system having different dimensions, weights, and power consumption. In some embodiments, the pocket-sized ultrasound system may provide the same functionality as the system 100 of FIG. 1.

In at least one embodiment discussed below, volumetric flow may be measured by integrating the flow flux through one or more imaging surfaces defined in a 3D data set or volume. The volume flow through a vessel or other anatomical structure is calculated without limiting the ultrasound data to a plane or planar surface formed or oriented parallel to the transducer face 138. The surface(s) may be arbitrarily shaped to allow for variations in vessel geometry and the transmitted ultrasound beams are adjusted or steered and/or the scanning parameters are otherwise modified so that the ultrasound beams are perpendicular to the surface(s) through which the flow is measured.

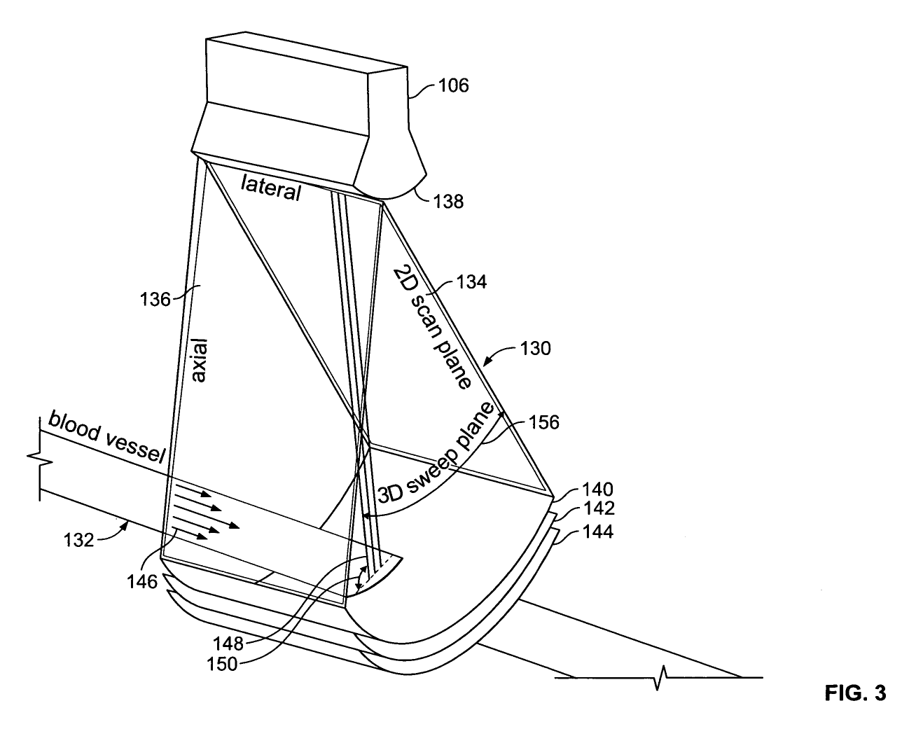

FIG. 3 illustrates an example of calculating volume of flow through a vessel based on at least two surfaces that are parallel to each other. Flow calculations through multiple isocentric or parallel surfaces that are defined through the same vascular structure may be averaged to provide a volume flow measurement that has improved signal to noise (S/N) characteristics. It should be noted that the multiple parallel surfaces are defined such that no vessel branching exists between the planes and that the volume flow is approximately the same between the multiple parallel surfaces (e.g., no significant blockage or narrowing of the vessel exists between the planes).

The probe 106 of FIG. 1 may be used to acquire volume 130 having vessel 132 therein. The volume 130 may comprise multiple scan planes such as a first scan plane 134 through N scan plane 136. For example, to acquire the volume 130, the probe 106 may electronically focus and direct ultrasound firings longitudinally over a 3D sweep plane 156 to scan along adjacent scan lines in each scan plane and electronically or mechanically focus and direct ultrasound firings, such as laterally, to scan adjacent scan planes. Scan planes may be scan converted from spherical to Cartesian coordinates. The orientation of the scan may be changed to sweep the 3D sweep plane 156 in a different direction.

Various embodiments may determine the volume of flow through the vessel 132 (while improving the S/N of the calculation). A first surface 140 and a second surface 142 are defined to fully intersect a cross-section of the vessel 132, such as by an operator using the user input 120 (as shown in FIG. 1). The first and second surfaces 140 and 142 may be defined as a plurality of points (not shown) wherein each of the points has a shortest distance to the transducer face 138. In this example, the first and second surfaces 140 and 142 are parallel with respect to each other and/or equidistant from each other. Optionally, additional surfaces, such as N surface 144, may be defined parallel to the first and second surfaces 140 and 142. The first through N surfaces 140-144 each represent a three-dimensional surface through at least a portion of the volume 130. Although the first through N surfaces 140-144 are illustrated as extending across the volume 130, the surfaces may be any size and shape as long as the surfaces bisect the vessel 132. Also, the first through N surfaces 140-144 do not need to be formed equidistant from the transducer face 138, but may be defined having a different orientation. For example, if the first surface 140 is formed equidistant from the transducer face 138, each of the plurality of points forming the surface has a same shortest distance to the transducer face 138.

Arrows 146 indicate a true velocity profile of blood flow within the vessel 132 and through the first through N surfaces 140-144. The processor 116 determines whether the first surface 140 is parallel to the transducer face 138 and thus receiving ultrasound beams at 90 degrees. If not, the transmission direction of each appropriate ultrasound beam(s) (or subset of ultrasound scan lines 148 that comprise the beam) are steered or directed to be perpendicular or 90 degrees with respect to the first surface 140. In this example, the scan lines 148 may be adjusted to bisect the first surface 140 (and thus the second through N surfaces 142 and 144) at a 90 degree angle 150. The volume of flow is then calculated through each of the first through N surfaces 140-144. The results may be averaged together to improve the S/N of the calculation.

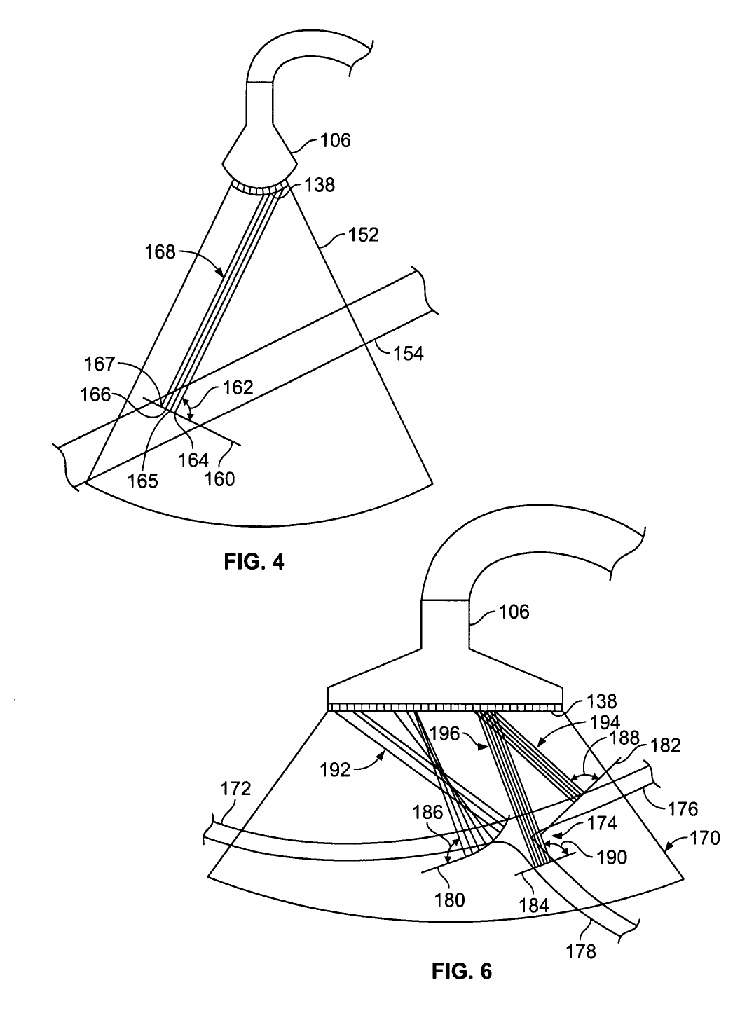

FIG. 4 illustrates an example of using a surface that is not parallel with respect to the transducer face to determine the amount of blood that is moving through a particular vessel. The probe 106 with the transducer face 138 is illustrated with scanned volume 152 having vessel 154 therein. The orientation of the probe 106 is known by the system 100, as is the transmission direction of the ultrasound energy used to acquire the volume 152.

The operator may use the user input 120 (FIG. 1) to define and subsequently modify surface 160. For example, to decrease the processing time or to optimize the processing speed, a diameter of the surface 160 may be decreased so that the surface 160 includes only the cross-section of the vessel 154, or the cross-section of the vessel 154 along with a small amount of surrounding tissue. Also, the surface 160 may be defined or modified to be any arbitrary shape, such as a curve that is either concave or convex, an uneven line, or a unique surface defined by the operator, such as to follow an anatomical structure.

In this example, the surface 160 is defined to extend across the vessel 154, but does not extend across the field of view of the volume 152. Additionally, the surface 160 is not parallel with respect to the transducer face 138. In other words, the surface 160 is defined as a series of points 164, 165, 166 and 167 that are unequal distances from the transducer face 138. It should be understood that limits of steering the ultrasound beams to intersect surface(s) at 90 degrees may exist. For example, a maximum angle may be determined by capabilities and geometry of the probe 106 beyond which the ultrasound beams may not be steered to achieve 90 degree intersection with respect to the surface.

The system 10 or 100 calculates the appropriate ultrasound beam formation to maintain the correct beam-to-surface orientation, and thus the ultrasound volume 152 or a portion of the volume 152, such as subset 168 of the ultrasound beams, is steered such that the ultrasound beams intersecting the surface 160 form a 90 degree angle 162. (Not all of the subsets 168 and/or ultrasound beams used to scan the surface 160 are shown.) If the user modifies the size, position, shape, orientation and the like of the surface 160, the system 100 calculates a new ultrasound beam formation. Therefore, a different subset of ultrasound beams may be used to scan the modified surface.

Scan apertures may also be used. For example, if a concave surface is defined that is relatively small with respect to the transducer face 138, a scan aperture may be moved to different positions on the transducer face 138 to intersect the concave surface at 90 degrees. Optionally, multiple scan apertures may be used to scan a single surface such as a concave or convex surface.

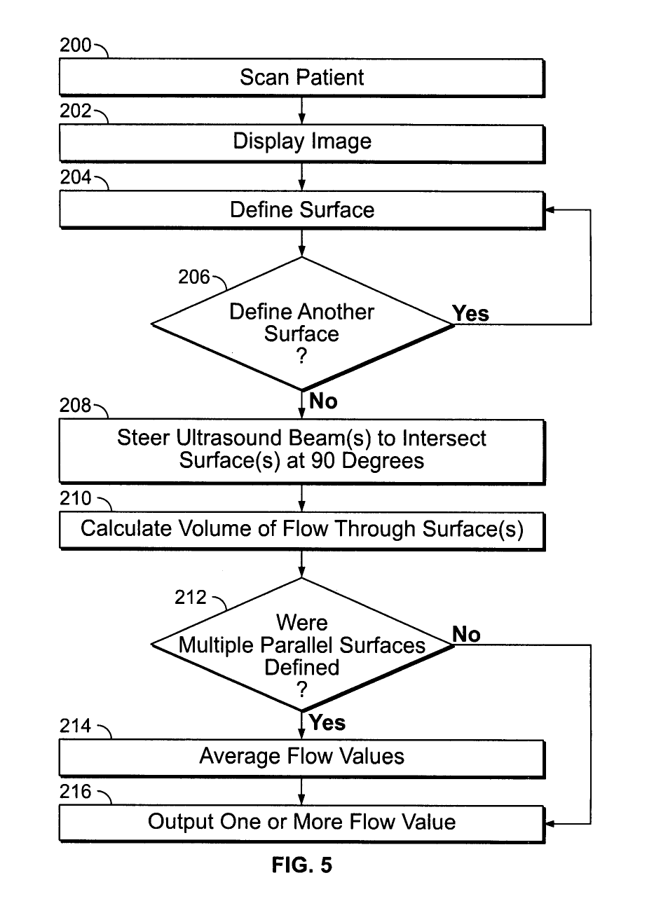

FIG. 5 illustrates a method for calculating volumetric flow information through a vessel. At 200, a volumetric scan of a patient is initiated. At 202, an image based on the volumetric scan is displayed, such as on the display 118 of FIG. 1. A complete diameter of the vessel(s) to be measured is within the displayed image. It may be desirable to display a volume, such as the volume 130 of FIG. 3, in 3D to ensure that the entire diameter of the vessel to be processed is within the volume 130. In other embodiments, a 2D image based on the volumetric scan may be displayed.

At 204, the operator defines a surface bisecting the vessel of interest. The operator may use the user input 120 (FIG. 1) and may change the location, shape, size and/or orientation of the surface as discussed previously. Turning to FIG. 4, the surface 160 is defined to extend through the vessel 154, but does not extend across the entire volume 152. Turning to FIG. 3, the first surface 140 may be defined to bisect the vessel 132 and extend across the entire volume 130. In another embodiment, the surface may be defined on a 2D image rather than a volume. Scanning in 3D may then be used to acquire the data associated with the surface wherein the 3D scanning may optionally not acquire data from a larger volume. At 206, if the operator wishes to define another surface, the method returns to 204. In the example of FIG. 3, the operator defines the second through N surfaces 142 and 144 as previously discussed.

FIG. 6 illustrates an example of using multiple surfaces to detect multiple volumetric flow values at different points within the same volume of data. The orientation of the surfaces may be determined by the location of multiple and/or branching vessels and the volume of flow may be simultaneously measured through more than one vessel location at a time. The probe 106 with transducer face 138 is illustrated along with acquired volume 170. A main vessel 172 is within the volume 170 and branches into first and second vessels 176 and 178 at vessel branching point 174.

Returning to 204 of FIG. 5, the operator defines first, second and third surfaces 180, 182, and 184 (as shown in FIG. 6) that bisect or extend through the main vessel 172 and first and second vessels 176 and 178, respectively. The first surface 180 is an irregularly shaped surface. The first, second and third surfaces 180, 182 and 184 may be defined at orientations that are not parallel to the transducer face 138.

At 208, the processor 116 and/or beamformer 110 determine the transducer elements 104 that will be used to transmit the ultrasound beams that intersect the surface(s) at 90 degrees. The transducer elements 104 may be steered such that the ultrasound beam(s), subset of transducer elements 104, or vectors are steered to intersect the surface(s) at 90 degrees and thus are normal to the surface(s). By way of example only, the ultrasound beams or lines of sight that are perpendicular to the surface, such that the surface covers the whole vessel cross-section, are determined, and the transducer elements 104 that symmetrically surround the location where the line of sight intersects the transducer face 138 are used. Optionally, the volumetric dataset may be segmented based on the surface(s). Referring to FIG. 3, the same ultrasound beams intersect the first through N surfaces 140-144 at a 90 degree angle 150 to detect the volume of flow through the first through N surfaces 140-144. Different ultrasound beams or subsets 192, 194 and 196 of transducer elements 104 may be steered in FIG. 6 to intersect the first, second and third surfaces 180, 182, and 184, respectively, at 90 degree angles 186, 188 and 190, respectively. As discussed previously, multiple apertures may be used to image the surface(s).

At 210, the processor 116 calculates the volume of flow through each surface in real-time, such as by integrating the 3D flow flux through each surface. At 212, if multiple parallel surfaces are defined at 204, such as in FIG. 3, the method continues to 214 where the processor 116 may average the multiple flow volume results from the multiple surfaces, providing a single volumetric flow calculation having improved S/N characteristics. The method continues to 216 from both 212 and 214, and the one or more volumetric flow values are output, such as by being displayed on the display 118. The output may be in the form of a number, graph, graphical indication, and the like (and may be provided in combination with a displayed image).

Volumetric flow may be further quantified by defining a boundary or edge of the vessel. The vessel cross-section comprises a middle area that is only vessel (e.g., blood flow region) and boundary or vessel edges that comprise a combination of both vessel and tissue (e.g., vessel wall). Once the surface is defined, such as at 204 of FIG. 5, the vessel boundary may be determined, such as by using power Doppler and/or B-flow. In B-flow, the flow and tissue data are simultaneously displayed as in B-mode. With power Doppler, the signal is smaller closer to the boundary. With B-flow, the strength of the flow signal decreases closer to the vessel wall.

Volumetric flow data detected in the middle of the vessel may be weighted by a predetermined amount, for example, 100 percent, while the volumetric flow data detected along the vessel edges is weighted by another amount, for example, a lesser amount. In other words, signals detected close to the vessel wall are weighted less than signals detected closer to the center of the vessel when determining the overall flow. Thus, the weighting may be varied across the cross-section of the vessel. Also, the rate of decrease of the power Doppler signal may be used to provide an indication of the weighting factor that should be applied to signal detected close to the boundary. Therefore, the lesser amount of weighting may be predetermined and/or may be based on the detected structure of the vessel.

A technical effect of at least one embodiment is the ability to calculate the volume of flow through a vessel within a 3D dataset based on a surface that extends through the vessel. The surface may not be parallel to the transducer face of the probe acquiring the 3D dataset. Beamforming and/or multiple apertures may be used to steer applicable ultrasound beam(s) to intersect the surface at 90 degrees. Multiple surfaces that are parallel to each other may be used to provide an average volume of flow having improved signal to noise characteristics. Also, multiple surfaces may be defined within a 3D dataset that are separate from each other. Each of the multiple surfaces may be oriented based on the anatomical structure rather than the transducer face, and thus may not be parallel to and/or equidistant with respect to the transducer face. By using multiple surfaces, volume flow through multiple vessels may be determined at the same time.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

What is claimed is:

1. An ultrasound system comprising:
an ultrasound probe comprising a transducer face emitting ultrasound beams into a patient, the probe acquiring a volume of ultrasound data comprising a blood vessel;
a user interface for defining a surface on an image that is based on the volume, the surface bisecting the blood vessel, the surface further comprising a plurality of points wherein at least some of the points are located at unequal distances with respect to the transducer face; and
a processor configured to control steering of a subset of the ultrasound beams to intersect the surface at a 90 degree angle, and calculate volumetric flow information through the blood vessel based on the ultrasound data corresponding to the surface.

2. The ultrasound system of claim 1, the user interface further defining a second surface bisecting the blood vessel, the first and second surfaces being parallel with respect to each other, the processor calculating second volumetric flow information through the blood vessel based on the ultrasound data corresponding to the second surface, the processor averaging the volumetric flow information and the second volumetric flow information.

3. The ultrasound system of claim 1, the user interface further defining a second surface intersecting the blood vessel, the processor steering a second subset of the ultrasound beams to intersect the second surface at a 90 degree angle, the processor calculating second volumetric flow information through the blood vessel based on the ultrasound data corresponding to the second surface.

4. The ultrasound system of claim 1, wherein the surface is one of a planar surface, a concave surface, a convex surface and an irregularly shaped surface.

5. The ultrasound system of claim 1, wherein the ultrasound system is one of a handheld, hand carried and portable system.

6. The ultrasound system of claim 1, wherein the user interface is configured to receive an input to adjust the surface with respect to the transducer face to position the surface at a second surface position, the processor steering a subset of the ultrasound beams to intersect the surface at the second surface position at a 90 degree angle.

7. A method for calculating volumetric flow information through a vessel, comprising:
acquiring a volume of ultrasound data with an ultrasound probe, the volume comprising a vessel, the ultrasound probe comprising a transducer face for emitting and receiving ultrasound beams;
defining first and second surfaces within the volume of ultrasound data, the first and second surfaces intersecting the vessel and being formed equidistant from each other; and
calculating an average volumetric flow through the vessel based on the ultrasound data corresponding to the first and second surfaces.

8. The method of claim 7, further comprising steering ultrasound beams associated with the first and second surfaces to intersect the first and second surfaces at a 90 degree angle.

9. The method of claim 7, wherein the first and second surfaces are formed parallel with respect to the transducer face.

10. The method of claim 7, wherein the first and second surfaces form at least one of a planar surface, a concave surface, a convex surface and an irregularly shaped surface.

11. The method of claim 7, further comprising:
defining a plurality of surfaces within the volume, each of the plurality of surfaces intersecting the vessel, each of the plurality of surfaces being formed parallel with respect to the first and second surfaces;
calculating a plurality of volumetric flows through the vessel based on the ultrasound data corresponding to the plurality of surfaces; and
averaging the average volumetric flow and the plurality of volumetric flows.

12. The method of claim 7, further comprising:
defining a third surface within the volume of ultrasound data, the third surface intersecting the vessel;
steering ultrasound beams associated with the third surface to intersect the third surface at a 90 degree angle; and
calculating a volumetric flow based on the ultrasound data corresponding to the third surface.

13. A method for calculating a volume of flow through a vessel, comprising:
acquiring a volume of ultrasound data with an ultrasound probe, the volume comprising a vessel, the ultrasound probe comprising a transducer face for emitting and receiving ultrasound beams;
defining a first surface on an image based on the volume, the first surface bisecting the vessel, the first surface further comprising a plurality of points where at least a portion of the points are at different distances from the transducer face;
steering a first subset of the ultrasound beams to intersect the first surface at a 90 degree angle; and
calculating a first volume of flow based on the ultrasound data corresponding to the first surface.

14. The method of claim 13, further comprising:
defining a second surface bisecting the vessel;
steering a second subset of the ultrasound beams to intersect the second surface at a 90 degree angle; and
calculating a second volume of flow through the vessel based on the ultrasound data corresponding to the second surface.

15. The method of claim 13, further comprising:
defining a second surface bisecting the vessel, the first and second surfaces being parallel with respect to each other;
calculating a second volume of flow through the vessel based on the ultrasound data corresponding to the second surface; and
averaging the first and second volumes of flow.

16. The method of claim 13, further comprising:
determining a vessel boundary of the vessel based at least on the ultrasound data corresponding to the surface; and
weighting the first volume of flow based on proximity to the vessel boundary.

17. The method of claim 13, further comprising:
imaging the first surface using at least one of power Doppler and B-flow imaging to detect signal strength across the vessel; and
weighting the first volume flow based on the signal strength, wherein a relatively larger weight corresponds to larger signal strength.

18. The method of claim 13, further comprising:
defining second and third surfaces bisecting the vessel;
steering second and third subsets of the ultrasound beams to intersect the second and third surfaces at 90 degree angles; and
calculating second and third volumes of flow through the vessel based on the ultrasound data corresponding to the second and third surfaces, respectively, the first, second and third volumes of flow being calculated simultaneously.

19. The method of claim 13, wherein the first surface extends across one of the image and a portion of the image comprising the vessel.

20. The method of claim 13, further comprising:
adjusting at least one of a location of the first surface within the image, a size of the first surface and a shape of the first surface; and
steering a subset of the ultrasound beams to intersect the first surface at a 90 degree angle.
